(19) United States
(12) Patent Application Publication
(10) Pub. No.: US 2013/0003912 A1
(43) Pub. Date: Jan. 3, 2013

(54) SYSTEM AND METHOD OF ACQUIRING COMPUTED TOMOGRAPHY DATA USING A MULTI-ENERGY X-RAY SOURCE

(75) Inventors: Bruno Kristiaan Bernard De Man, Clifton Park, NY (US); Jiang Hsieh, Brookfield, WI (US); Naveen Stephan Chandra, Waukesha, WI (US); Jiahua Fan, New Berlin, WI (US); Jed Douglas Pack, Glenville, NY (US)

(73) Assignee: General Electric Company, Schenectady, NY (US)

(21) Appl. No.: 13/174,485

(22) Filed: Jun. 30, 2011

(51) Int. Cl. A61B 6/03; G01N 23/04

(52) U.S. Cl. 378/5; 378/16

(57) ABSTRACT

The subject matter disclosed herein relates to X-ray imaging systems, and more specifically, to multi-energy computed tomography (CT) X-ray imaging systems. In an embodiment, a multi-energy computed tomography (CT) imaging system includes an X-ray source that emits X-rays upon the application of a low stable bias, a high stable bias, and transitional biases between the low stable bias and the high stable bias. The imaging system also includes an X-ray detector configured to produce an electrical signal corresponding to the intensity of the X-rays emitted by the X-ray source that reach the X-ray detector. The imaging system also includes data processing circuitry configured to acquire a first set of data corresponding to the electrical signal produced by the X-ray detector only when the low stable bias or the high stable bias is applied to the X-ray source. The imaging system also includes a processor configured to process the first set of acquired data and construct one or more multi-energy CT images.

### BACKGROUND

In modern medicine, medical professionals routinely desire to conduct patient imaging examinations to assess the internal tissue of a patient in a non-invasive manner. For typical single-energy computed tomography (CT) imaging, the resulting X-ray images are largely a representation of the average density of each analyzed voxel based upon the attenuation of X-rays between the X-ray source and the X-ray detector by the patient tissue. However, for multi-energy X-ray imaging a greater amount of imaging data may be gleaned for each voxel. For example, in a dual-energy X-ray imaging system, X-rays of two different energies (i.e., different frequencies) are employed, and the higher energy X-rays generally interact substantially less with patient tissue than the lower energy X-rays. In order to reconstruct multi-energy CT projection data, the underlying physical effects of the X-rays are discerned, namely, the scattering effects and photoelectric effects, in a process known as material decomposition (MD).

During multi-energy CT data acquisition, a multi-energy X-ray source may be used to provide the X-rays having different energies and may be capable of quickly switching from emitting X-rays having one average energy to emitting X-rays having a different average energy (i.e., a fast-switching source). For example, the X-ray source may be an X-ray tube, and by modulating the applied bias between a lower voltage and a higher voltage (e.g., several times per second), X-rays having a higher energy and a lower energy may be emitted. However, a fast switching multi-energy X-ray source may also emit X-rays having intermediate energies as the source is switching between emitting X-rays having two different energies. That is, for example, while a dual-energy source may be configured to emit X-rays of a particular lower energy when a lower voltage bias is applied and X-rays of a higher energy when a higher voltage bias is applied, in practice the source may also emit X-rays having energies between the lower and higher energies as the bias being applied to the source is switching between the lower and higher voltage biases.

In reconstructing the projection data acquired during multi-energy CT imaging, attempting to discern the scattering effects from the photoelectric effects using a MD computation becomes increasingly difficult and computationally costly when the energies of the X-rays are not clearly resolved. That is, it may be impractical to computationally separate these physical effects when a continuum of X-ray energies are actually being presented to the X-ray detector rather than just X-rays having two or more well-resolved energies.

### BRIEF DESCRIPTION

In an embodiment, a multi-energy computed tomography (CT) imaging system includes an X-ray source that emits X-rays upon the application of a low stable bias, a high stable bias, and transitional biases between the low stable bias and the high stable bias. The imaging system also includes an X-ray detector configured to produce an electrical signal corresponding to the intensity of the X-rays emitted by the X-ray source that reach the X-ray detector. The imaging system also includes data processing circuitry configured to acquire a first set of data corresponding to the electrical signal produced by the X-ray detector only when the low stable bias or the high stable bias is applied to the X-ray source. The imaging system also includes a processor configured to process the first set of acquired data and construct one or more multi-energy CT images.

In an embodiment, a multi-energy radiation imaging system includes a radiation source that emits radiation through the application of two or more stable biases and corresponding unstable biases between each stable bias. The imaging system also includes a radiation detector configured to receive the radiation from the radiation source and to produce an electrical signal corresponding to the intensity of the received radiation. The imaging system also includes data processing circuitry configured to acquire a first set of data from the electrical signal produced by the radiation detector when an activation signal is supplied and configured to acquire a second set of data from the electrical signal produced by the radiation detector when the activation signal is not supplied. The imaging system also includes a controller unit coupled to the radiation source and the data processing circuitry and configured to synchronize the application of the stable biases to the radiation source with the application of the activation signal to the data processing circuitry.

In an embodiment, a method of improving energy separation in a multi-energy switching X-ray imaging system includes monitoring a source bias of a switching X-ray source as it is switched between a low bias and a high bias and emits X-rays. The method also includes detecting the emitted X-rays using an X-ray detector that produces an electrical signal corresponding to the detected X-rays. The method also includes activating data processing circuitry to acquire a first set of data from the detector when the source bias is stable at the low bias or at the high bias. The method also includes processing the first set of acquired data with a processor to construct one or more multi-energy computer tomography (CT) images.

### BRIEF DESCRIPTION OF THE DRAWINGS

These and other features and aspects of embodiments of the present invention will become better understood when the following detailed description is read with reference to the accompanying drawings in which like characters represent like parts throughout the drawings, wherein:

FIG. 1 illustrates an embodiment of a multi-energy CT imaging system, in accordance with aspects of the present disclosure;

FIG. 2 illustrates a plot of the bias applied to an embodiment of a multi-energy X-ray source over time, in accordance with aspects of the present disclosure;

FIG. 3 illustrates a plot of the bias applied to an embodiment of another multi-energy X-ray source over time, in accordance with aspects of the present disclosure; and

FIG. 4 depicts a flow diagram illustrating a process by which the patient imaging system acquires and processes X-ray projection data, in accordance with aspects of the present disclosure.

### DETAILED DESCRIPTION

The disclosed embodiments illustrate a method of separately processing the X-ray projection data acquired when a multi-energy X-ray source is emitting X-rays of stable energy levels and X-ray projection data acquired when the X-ray source is emitting X-rays of intermediate energy levels. The term “stable energy levels”, in the context of the present application, refers to energy levels of X-rays that the X-ray source may be capable of emitting for a period of time without significant variation. That is, for example, a dual-energy X-ray source may be capable of switching between emitting X-rays of a high stable energy level and a low stable energy level. Accordingly, the terms “intermediate energy levels” or “unstable energy levels” refer to the energy levels of X-rays that lie between stable energy levels. Likewise, for a bias driven X-ray source, the terms “stable bias” or “stable voltage”, in the context of the present application, refer to a bias that may be applied to the X-ray source for period without substantial variation such that X-rays of a stable energy level are emitted. Similarly, the terms “intermediate bias or voltage”, “unstable bias or voltage”, or “transitional bias or voltage” refer to a bias applied to the X-ray source that is between stable biases or voltages (e.g., when in transition between stable biases) and is capable of producing X-rays of an intermediate energy level. As such, the term “transition period” refers to the time window in which the X-ray source is receiving an unstable bias and/or emitting X-rays of unstable energy levels, while a “stable period” refers to a time window in which the X-ray source is receiving a stable bias and/or emitting X-rays of stable energy levels.

In general, the disclosed embodiments include different methods of separately processing X-ray projection data acquired using X-rays of stable energy levels and projection data acquired during transition periods when the X-rays are of unstable or intermediate energy levels. In certain embodiments, the X-ray source may be configured to not emit X-rays when receiving an unstable bias during transition periods, and therefore, X-ray projection data is only acquired during the emission of X-rays of stable energy levels. In other embodiments, the projection data that is acquired using X-rays of unstable energy levels may be ignored or discarded, leaving only the projection data acquired using X-rays of stable energy levels to be used for the MD reconstruction of multi-energy CT image(s). In certain embodiments, the projection data acquired during the transition window may be used to reconstruct non-multi-energy CT images (e.g., regular or monochromatic CT images), while only projection data acquired when the source is emitting X-rays of stable energy levels may be used for the MD reconstruction of multi-energy CT image(s). In certain embodiments, projection data may acquired from X-rays of both stable and unstable energy levels, and the MD reconstruction process may rely upon a weighted estimator that may enable the projection data acquired from X-rays of stable energy levels to receive a greater weight in the MD computation.

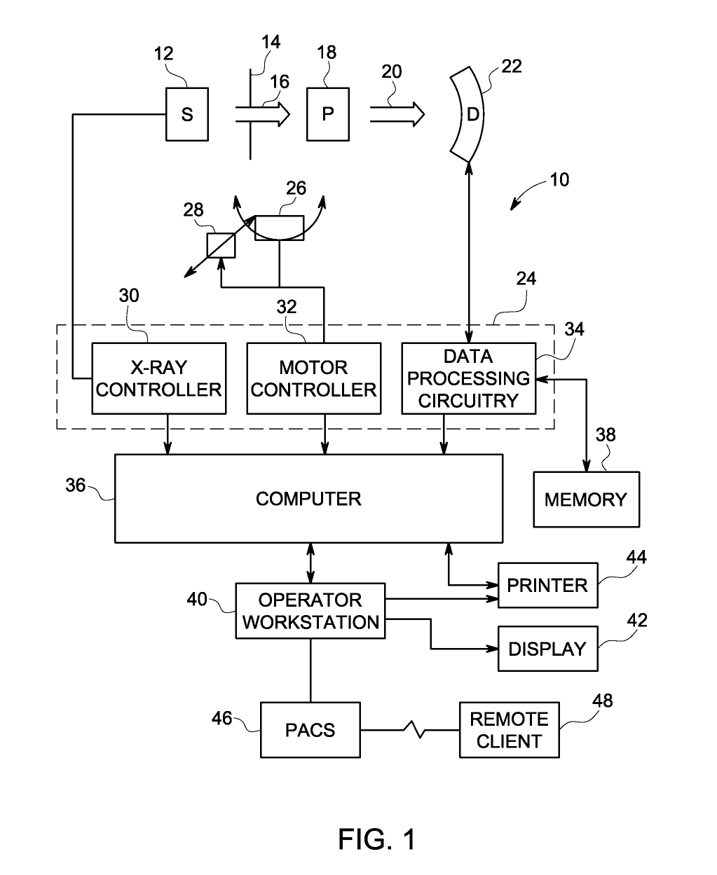

With the forgoing discussion in mind, FIG. 1 illustrates diagrammatically an imaging system 10 for acquiring and processing projection data. In the illustrated embodiment, system 10 is a multi-energy computed tomography (CT) system designed to acquire multi-energy and non-multi-energy X-ray projection data, to reconstruct the projection data into an image, and to process the image data for display and analysis in accordance with the present technique. Though the imaging system 10 is discussed in the context of medical imaging, the techniques and configurations discussed herein are applicable in other non-invasive imaging contexts, such as baggage or package screening. In the embodiment illustrated in FIG. 1, multi-energy CT imaging system 10 includes a source 12 of X-ray radiation. As discussed in detail herein, the source 12 of X-ray radiation is a multi-energy X-ray source, such as an X-ray tube, or a distributed source configured to emit X-rays from different locations along a surface. For example, the multi-energy X-ray source 12 may include one or more addressable solid-state emitters. Such solid-state emitters may be configured as arrays of field emitters, including one-dimensional arrays, i.e., lines, and two-dimensional arrays. The multi-energy X-ray source is configured to emit X-rays of two or more stable energy levels. For example, a multi-energy source may be capable of emitting X-rays of 2, 3, 4, or 5 different stable energy levels upon application of 2, 3, 4, or 5 different stable voltages.

The multi-energy X-ray source 12 may be positioned proximate to a collimator 14. The collimator 14 may consist of one or more collimating regions, such as lead or tungsten shutters, for each emission point of the source 12. The collimator 14 typically defines the size and shape of the one or more beams of radiation 16 that pass into a region in which a subject, such as a human patient 18 is positioned. A beam of radiation 16 may be generally fan-shaped or cone-shaped, depending on the configuration of the detector array. An attenuated portion of the radiation 20 passes through the subject, which provides the attenuation, and impacts a detector array, represented generally at reference numeral 22.

The detector 22 is generally formed by a plurality of detector elements, which detect the X-rays that pass through and around a subject of interest. Each detector element produces an electrical signal that represents the intensity of the X-ray beam incident at the position of the element during the time the beam strikes the detector. Typically, signals are acquired at a variety of angular positions around the subject of interest so that a plurality of radiographic views may be collected. These signals are acquired and processed to reconstruct an image of the features within the subject, as described below.

The multi-energy X-ray source 12 is controlled by a system controller 24, which furnishes power, focal spot location, control signals and so forth for CT examination sequences. Moreover, the detector 22 is coupled to the system controller 24, which commands acquisition of the signals generated in the detector 22. The system controller 24 may also execute various signal processing and filtration functions, such as for initial adjustment of dynamic ranges, interleaving of digital image data, and so forth. In general, system controller 24 commands operation of the imaging system to execute examination protocols and to process acquired data. In the present context, system controller 24 also includes signal processing circuitry and associated memory circuitry. The associated memory circuitry may store programs and routines executed by the system controller, configuration parameters, image data, and so forth. In one embodiment, the system controller 24 may be implemented as all or part of a processor-based system such as a general purpose or application-specific computer system.

In the embodiment illustrated in FIG. 1, system controller 24 may control the movement of a linear positioning subsystem 28 and rotational subsystem 26 via a motor controller 32. In imaging systems 10 in which the source 12 and/or the detector 22 may be rotated, the rotational subsystem 26 may rotate the X-ray source 12, the collimator 14, and/or the detector 22 through one or multiple turns around the patient 18. It should be noted that the rotational subsystem 26 may include a gantry. The linear positioning subsystem 28 enables the patient 18, or more specifically a patient table, to be displaced linearly. Thus, the patient table may be linearly moved within the gantry or within the imaging volume defined by source 12 and/or detector 22 configuration to generate images of particular areas of the patient 18. In embodiments comprising a stationary source 12 and a stationary detector 22, the rotational subsystem 26 may be absent. Similarly, in embodiments in which the source 12 and the detector 22 are configured to provide extended or sufficient coverage along the Z-axis, i.e, the axis associated with the main length of the patient 18, the linear positioning subsystem 28 may be absent.

Further, the system controller 24 may comprise data processing circuitry 34. In this embodiment, the detector 22 is coupled to the system controller 24, and more particularly to the data processing circuitry 34. The data processing circuitry 34 receives data collected by the detector 22. The data processing circuitry 34 typically receives sampled analog signals from the detector 22 and converts the data to digital signals for subsequent processing by a processor-based system, such as a computer 36. Alternatively, in other embodiments, the detector 22 may include a digital-to-analog converter to convert the sampled analog signals to digital signals prior to transmission to the data processing circuitry 34. Additionally, in certain embodiments, the data processing circuitry 34 that may be selectively activated by the system controller 24 (e.g., via activation signals) to receive signals from the detector 22.

Additionally, the multi-energy X-ray source 12 may be controlled by an X-ray controller 30 disposed within the system controller 24. The X-ray controller 30 may be configured to provide power and timing signals to the X-ray source 12. For example, the X-ray controller 30 may include a fast-switching power supply configured to supply the source 12 with at least two or more stable biases to produce X-rays of two or more stable energy levels. Additionally, the X-ray controller 30 may also include sensing and processing circuitry configured to monitor the source bias as well as compute and store statistical information (e.g., average or mean stable biases, average period for the source bias curve, etc.) for determining the stability of the bias at a point in time, as discussed in detail below. Furthermore, the X-ray controller 30 may supply system controller 24 with information regarding the source bias at a point in time (e.g., stable bias versus unstable bias) as well as the statistical information regarding the source bias curve. With this information, the system controller 24 may identify transition periods to determine whether detected X-rays were emitted from a stable or unstable applied bias, and therefore, whether the detected X-rays are of stable or unstable energy levels. As discussed in detail below, this may allow the projection data acquired during the transition periods to be processed separately from the remainder of the data. In certain embodiments, the X-ray controller 30 may also be configured supply a gating signal to the X-ray source 12 that may prevent the X-ray source from emitting when applied, as discussed in detail below.

Alternatively, in certain embodiments, the system controller 24 may include a clock (e.g., a time processing unit) such that the activities of the components of the CT imaging system 10 may be synchronized. For example, clock may provide signals to allow the system controller 24 to correlate in time the application of a stable bias (e.g., a lower stable bias, a higher stable bias, etc.) to the source 12 with the activation of the data processing circuitry 34 (e.g., via an activation signal) to acquire data from the detector 22. In certain embodiments, the clock may provide signals to also correlate in time the application unstable biases to the source 12 with the deactivation of the data processing circuitry 34 (e.g., via a deactivation signal or a cessation of the activation signal) to acquire data from the detector 22.

In the depicted embodiment, the computer 36 is coupled to the system controller 24. The data collected by the data processing circuitry 34 may be transmitted to the computer 36 for subsequent processing and reconstruction. The computer 36 may comprise or communicate with a memory 38 that can store data processed by the computer 36, data to be processed by the computer 36, or routines to be executed by the computer 36, such as for processing image data in accordance with the present technique. It should be understood that any type of computer accessible memory device capable of storing the desired amount of data and/or code may be utilized by such a system 10. Moreover, the memory 38 may comprise one or more memory devices, such as magnetic or optical devices, of similar or different types, which may be local and/or remote to the system 10. The memory 38 may store data, processing parameters, and/or computer programs comprising one or more routines for performing the processes described herein.

The computer 36 may also be adapted to control features enabled by the system controller 24, i.e., scanning operations and data acquisition. Furthermore, the computer 36 may be configured to receive commands and scanning parameters from an operator via an operator workstation 40 which may be equipped with a keyboard and/or other input devices. An operator may thereby control the system 10 via the operator workstation 40. Thus, the operator may observe the reconstructed image and other data relevant to the system from computer 36, initiate imaging, select and apply image filters, and so forth. Further, the operator may manually identify features and regions of interest from the reconstructed image or the operator may review features and regions of interest automatically identified and/or enhanced through computer-aided geometry determination as discussed herein. Alternatively, automated detection algorithms may be applied to such enhanced features or regions of interest.

A display 42 coupled to the operator workstation 40 may be utilized to observe the reconstructed image. Additionally, the reconstructed image may be printed by a printer 44 which may be coupled to the operator workstation 40. The display 42 and printer 44 may also be connected to the computer 36, either directly or via the operator workstation 40. Further, the operator workstation 40 may also be coupled to a picture archiving and communications system (PACS) 46. It should be noted that PACS 46 might be coupled to a remote system 48, radiology department information system (RIS), hospital information system (HIS) or to an internal or external network, so that others at different locations may gain access to the image data.

One or more operator workstations 40 may be linked in the system for outputting system parameters, requesting examinations, viewing images, and so forth. In general, displays, printers, workstations, and similar devices supplied within the system may be local to the data acquisition components, or may be remote from these components, such as elsewhere within an institution or hospital, or in an entirely different location, linked to the image acquisition system via one or more configurable networks, such as the Internet, virtual private networks, and so forth.

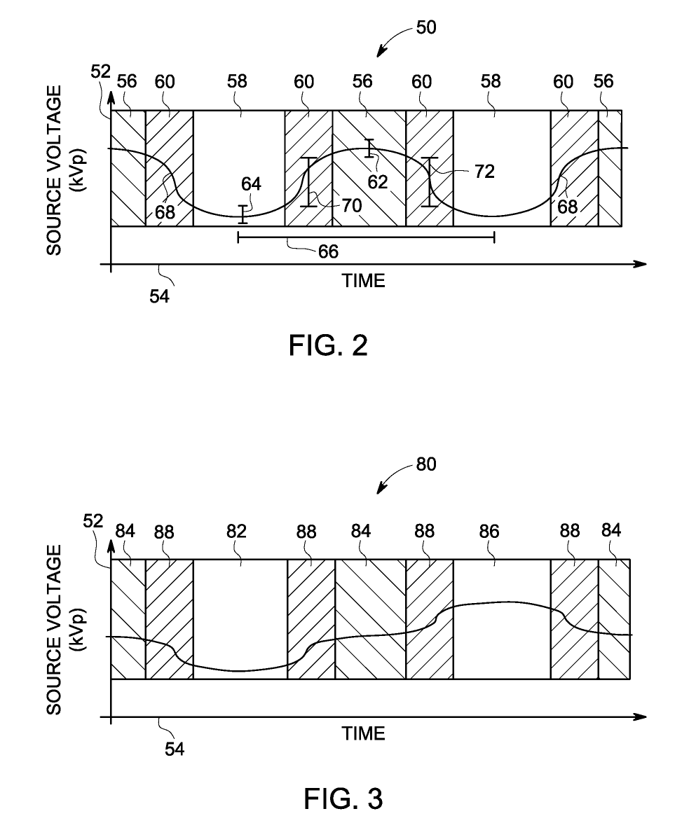

As discussed above, the X-ray controller 30 may supply and monitor the bias being applied to the multi-energy X-ray source. For example, FIG. 2 illustrates a plot 50 of the source voltage 52 (i.e., the bias being applied to an X-ray source) over time 54. In the illustrated embodiment, the source bias oscillates approximately sinusoidally between higher stable voltage regions 56 and lower stable voltage regions 58 with transition periods 60 having intermediate unstable voltages between each. For example, the source bias may be approximately 140 kVp for the higher stable voltage regions 56, while the source bias may be approximately 80 kVp for the lower stable voltage regions 58.

In FIG. 2, some variation in source bias may be observed near the edges of the illustrated higher and lower stable bias regions. In certain embodiments, the source bias may be considered stable by the X-ray controller 30 when the source bias change (or drift) is less than approximately 10% for a length of time. In certain implementations, the length of time may be on the order of the acquisition time interval of the detector 22 or a multiple thereof. For example, for an X-ray detector 22 having an acquisition time interval of approximately 350 μs, the source bias may be considered to be stable by the X-ray controller 30 when it changes by less than approximately 10% over 350 μs or 700 μs. In certain embodiments, the length of time may be a fraction (or a percentage) of the period 66 (i.e. the duration of one cycle) of the source bias curve. For example, if the period 66 of the source bias curve is approximately 1 ms, X-ray controller 30 may consider the source bias as stable when it changes by less than approximately 10% over 500 μs (i.e., 50% of the period 66) or 250 μs (i.e., 25% of the period 66). In other embodiments, the X-ray controller 30 may determine a mean stable bias, and a source bias may be considered stable when it is within approximately 10% of the mean stable bias (as illustrated by ranges 62 and 64). For example, for an X-ray source having a mean higher stable bias of 140 kVp, the X-ray controller may consider the source bias as stable when it is at 140 kVp±10% (i.e., between 154 kVp and 126 kVp).

The transition periods 60 of the illustrated embodiment encompass the portions of the source bias curve experiencing the greatest fluctuation in source voltage (i.e., the inflection points 68). In certain embodiments, the source bias may be considered unstable by the X-ray controller 30 when the source bias change or drift is greater than approximately 10% for a length of time. In certain implementations, the length of time may be the acquisition time interval of the detector 22 or a multiple thereof. For example, for an X-ray detector 22 having an acquisition time interval of approximately 400 μs, the source bias may be considered to be unstable by the X-ray controller 30 when it changes by more than approximately 10% over approximately 400 μs or 800 μs. In certain embodiments, the length of time may be a fraction or a percentage of the period 66 of the source bias curve. For example, if the period 66 of the source bias curve is approximately 1 ms, X-ray controller 30 may consider the source bias as unstable when it changes by greater than approximately 10% over approximately 350 μs (i.e., 35% of the period) or 100 μs (i.e., 10% of the period). In certain embodiments, the X-ray controller 30 may determine the mean stable biases for the X-ray source 12 and consider the source bias unstable when it is not within 10% of a mean stable bias (as illustrated by ranges 70 and 72). For example, if the mean stable biases of an X-ray source were approximately 80 kVp and 140 kVp, the X-ray controller 30 may consider any source biases in the range between approximately 89 kVp (i.e., >80 kVp+10%) and 125 kVp (i.e., <140 kVp−10%) as unstable.

With this in mind, one embodiment of a multi-energy CT imaging system 10 may include a system controller 24 and the data processing circuitry 34 that do not collect the projection data during transition periods 60. In general, as discussed above, this may be desirable for multi-energy CT MD reconstructions since the projection data acquired during the transition periods 60 may not contribute much to the signal or contrast data but may substantially contribute to the noise level. In one embodiment, the system controller 24 may rely upon the information provided by the X-ray controller 30 regarding the bias being presently applied to the multi-energy X-ray source 12 in order to synchronize the activation of the detector 22 and/or the data processing circuitry 34 with the application of a stable bias to the source 12 (e.g., stable higher bias regions 56 or stable lower bias regions 58). In certain embodiments, the system controller 24 may instead deactivate the detector 22 and/or the data processing circuitry 34 during the transition periods 60 so that no projection data is collected. In other embodiments, the detector 12 and the data processing circuitry 34 may remain active to acquire projection data during the transition periods 60, and then the projection data collected from the transition periods may be subsequently discarded.

However, rather than not collecting or discarding projection data during the transition periods 60, it may be desirable to prevent the multi-energy X-ray source 12 from emitting X-rays during the transition periods 60. As such, certain embodiments may include a gated multi-energy X-ray source 12 that may be controlled by a gate signal from the X-ray controller 30. For example, the X-ray source 12 may be an X-ray tube including a cathode and an anode, across which the X-ray controller applies a voltage (e.g., the source bias curve 50) in order to produce X-rays. In addition, a gated X-ray source may also include, for example, a filter or screen disposed near the cathode that may be biased (e.g, via the gating signal). By placing a bias on the filter, electrons leaving the cathode are attracted to, or repulsed by, the filter such that they do not arrive at the anode, and therefore, X-rays may not be emitted. In such an embodiment, the gating signal may be synchronized with the transition periods 60 such that X-rays may not be emitted, and therefore, projection data may only be acquired for the stable bias regions 56 and 58 when X-rays are emitted.

In another embodiment, it may be desirable to collect projection data during the stable bias regions (56 and 58) as well as the transition periods 60, and then process the data separately. That is, while the projection data acquired during the transition periods 60 may be problematic when included in the MD reconstruction process, the projection data acquired during transition periods 60, alone or in combination with projection data acquired during stable periods 56 and 58, may still be useful for producing other types of X-ray images (e.g., non-multi-energy or monochromatic CT images). Therefore, in an embodiment, the data processing circuitry 34 may be used to acquire projection data during both the transition periods 60 and the stable periods 56 and 58. In such an embodiment, the data processing circuitry 34 may store the projection data acquired during the stable regions (56 and 58) separately from projection data acquired during the transition periods 60 (e.g., in separate bins, separate spaces in memory 38, or separate memory or storage spaces in computer 36) for separate processing by the computer 36.

In another embodiment, it may be desirable to collect and store the projection data from both the stable bias regions (58 and 60) and the transition regions 60, as described above, and include all of the acquired projection data in the MD reconstruction process, giving a greater computational weight to projection data acquired during stable bias regions 58 and 60. For example, the MD reconstruction process may use a weighted estimator (e.g., a weighted least squares estimate, weighted average, or weighted subtraction), and the stable bias regions 58 and 60 may receive a higher weight since they contain more of the information for the MD reconstruction process. The projection data acquired during the transition regions 60 may receive a lower weight since it may contribute some information for monochromatic sonograms but much less for the material decomposition sonograms. Alternatively, in certain embodiments, a weighted subtraction may be used, and the projection data acquired during the transition periods may receive a higher weight than the data acquired during the stable periods and, therefore, may have less effect on the MD reconstruction. Indeed, even within the projection data acquired during a stable region (e.g., 58 or 60) or a transition region 60, the projection data may be weighted differently. For example, the projection data acquired during from the middle of a transition period 60 may receive a different weight than the projection data acquired near the beginning or end of the transition period 60. Accordingly, for any weighted technique, by adjusting the weight that portions of the projection data in the stable regions (58 and 60) and transition regions 60 receive in the computation, the aforementioned deleterious effects of the noise introduced into MD reconstruction process by the projection data from the transition periods may be mitigated.

FIG. 3 further illustrates a plot 80 of the source bias 52 over time 54 for an embodiment of a multi-energy X-ray source 12 having three stable biases and, accordingly, is capable of emitting X-rays of three different stable energy levels. In the illustrated embodiment, the three stable biases regions include a low stable bias region 82, medium stable bias region 84, and high stable bias region 86 with unstable bias regions 88 (i.e., transition periods) between each. For an embodiment of an X-ray source having three stable biases 82, 84 and 86, similar to the previously described embodiments having two stable biases 56 and 58, the transition periods 88 may be addressed in similar ways.

For example, in certain embodiments, the X-ray source 12 may be configured to not emit X-rays when receiving an unstable bias during transition periods 88 (e.g., via a gating signal supplied to a filter by the X-ray controller 30), and therefore, projection data may only be acquired during the stable bias regions 82, 84 and 86. In other embodiments, the projection data acquired during transition periods 88 may be ignored (e.g., via deactivation of the detector 22 and/or data processing circuitry 34) or collected and discarded so that only the projection data acquired during stable periods 82, 84, and 86 may be used for material decomposition computation. In certain embodiments, the projection data acquired during the stable bias regions 82, 84, and 86 and/or the transition period 88 may be used to construct non-multi-energy CT images (e.g., regular or monochromatic CT images), while only projection data acquired from stable bias regions 82, 84, and 86 may be included in the MD reconstruction of the multi-energy CT image. In certain embodiments, projection data may be acquired during stable bias regions 82, 84, and 86 as well as the transition periods 88, and the MD reconstruction process may rely upon a weighted estimator (e.g., a weighted least squares estimate) that may allow the projection data acquired from X-rays of stable energy levels (i.e., during stable bias regions 82, 84, and 88) to receive a greater weight in the MD reconstruction.

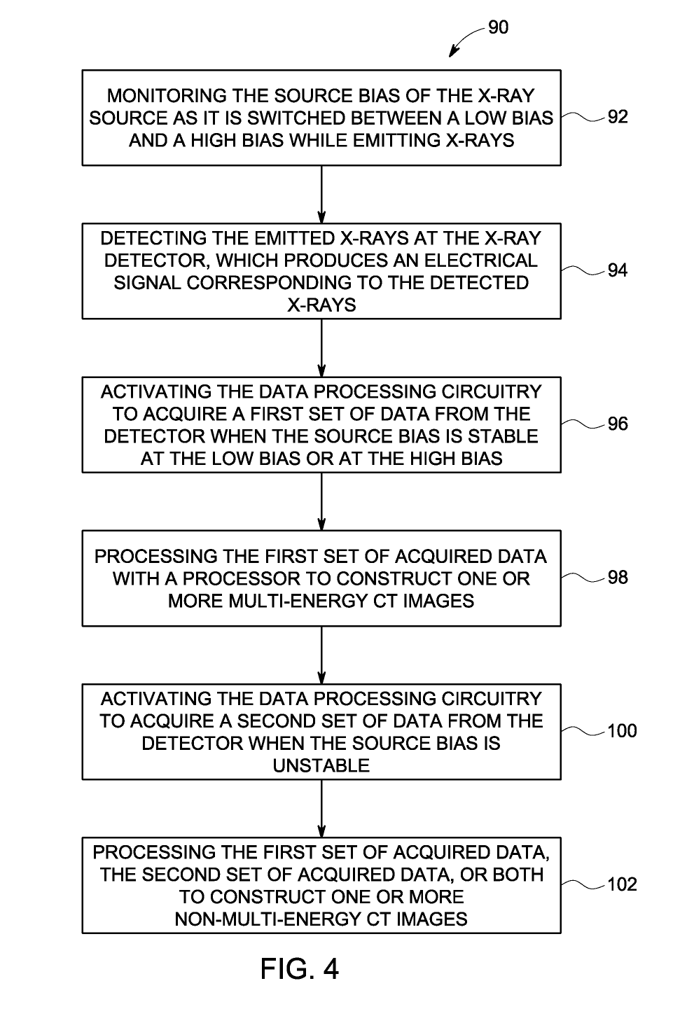

FIG. 4 illustrates an embodiment of a process 90 by which the patient imaging system 10 may be used to acquire sets projection data during periods of stable and unstable source biases and to process the sets of acquired projection data appropriately to construct different types of CT images. The process 90 begins with the patient imaging system 10 monitoring (block 92) the bias applied to the X-ray source 12 as it is switched between a low bias and a high bias while emitting X-rays. For example, the X-ray controller 30 and/or the system controller 24 may monitor the source bias as it is switched between approximately 80 kVp and 140 kVp. Next, the patient imaging system 10 may detect (block 94) the emitted X-rays at the X-ray detector 22, which produces an electrical signal corresponding to the detected X-rays. The patient imaging system 10 (e.g., the system controller 24 of the system 10) may then activate (block 96) the data processing circuitry 34 to acquire a first set of projection data from the detector 22 when the source bias is stable at the low bias or the high bias. For example, when the system controller 24 is monitoring the source bias and determines that the source bias is within approximately 10% of the mean stable high or low bias (e.g., within 10% of 80 kVp or 140 kVp), the system controller 24 may activate the data processing circuitry 34 to acquire a first set of projection data from the detector 22. After the first set of projection data has been acquired, the patient imaging system 10 may process (block 98) the first set of projection data with a processor (e.g., computer 36) to construct one or more multi-energy CT images.

The patient imaging system 10 may also activate (block 100) the data processing circuitry 34 to acquire a second set of projection data from the detector 22 when the source bias is unstable. For example, when the system controller 24 is monitoring the bias being applied to the X-ray source 12 and determines that the source bias is not within approximately 10% of the mean stable low or high bias (e.g., not within 10% of 80 kVp or 140 kVp), the system controller 24 may activate the data processing circuitry 34 to acquire a second set of projection data from the detector 22. In certain embodiments, the acquisition of the second set of projection data (block 100) need not wait for the completion of the processing of the first set of acquired data (block 98) before beginning, allowing these steps to be performed in parallel.

After the second set of projection data has been acquired, the patient imaging system 10 may process (block 102) this first set of projection data, the second set of projection data, or both, with a processor (e.g., computer 36) to construct one or more non-multi-energy CT images. As discussed above, in other embodiments, the patient imaging system may deactivate the data processing circuitry 34 and/or the detector during unstable bias periods, prevent the X-ray source 12 from emitting during unstable bias periods, and/or collect and discard projection data acquired during unstable bias periods; therefore, for such embodiments, the final two steps (blocks 100 and 102) of the process 90 may not be performed.

Technical effects of the invention include reducing the computational time and difficulty for performing a material decomposition reconstruction of multi-energy CT projection data. By treating the transition periods differently from the stable bias regions, the quality of the projection data used for the MD reconstruction process may be improved. Additionally, in some embodiments, by collecting the projection data from the transition periods for separate processing into non-multi-energy CT images or for incorporation into the MD reconstruction using a weighted calculation, the imaging process may insure that the acquired projection data is used in an effective manner. Furthermore, in some embodiments, by preventing the X-ray source from emitting during transition periods, the patient may be exposed to less radiation during the exam.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

1. A multi-energy computed tomography (CT) imaging system comprising:
an X-ray source that emits X-rays upon the application of a low stable bias, a high stable bias, and transitional biases between the low stable bias and the high stable bias;
an X-ray detector configured to produce an electrical signal corresponding to the intensity of the X-rays emitted by the X-ray source that reach the X-ray detector;
data processing circuitry configured to acquire a first set of data corresponding to the electrical signal produced by the X-ray detector only when the low stable bias or the high stable bias is applied to the X-ray source; and
a processor configured to process the first set of acquired data and construct one or more multi-energy CT images.

2. The system of claim 1, wherein the X-ray source comprises a filter that is configured to allow the X-ray source to only emit X-rays when the low stable bias or the high stable bias is applied to the X-ray source.

3. The system of claim 1, comprising a clock configured to provide signals to correlate in time the application of the low stable bias or the high stable bias to the X-ray source and the acquisition of the first set of data by the data processing circuitry.

4. The system of claim 1, wherein the data processing circuitry is configured to acquire a second set of data corresponding to the electrical signal produced by the X-ray detector when the transitional biases between the low stable bias and the high stable bias are applied to the X-ray source.

5. The system of claim 4, comprising a clock configured to:
provide signals to correlate in time the application of the low stable bias or the high stable bias to the X-ray source and the acquisition of the first set of data by the data processing circuitry; and
provide signals to correlate in time the application of the transitional biases to the X-ray source and the acquisition of the second set of data by the data processing circuitry.

6. The system of claim 4, wherein the processor is configured to process the first set of acquired data, the second set of acquired data, or both, to construct non-multi-energy CT images.

7. The system of claim 4, wherein the processor uses the first set of acquired data and the second set of acquired data to construct a multi-energy CT image using a weighted calculation in which the first set of acquired data is weighted differently than the second set of acquired data.

8. A multi-energy radiation imaging system comprising:
a radiation source that emits radiation through the application of two or more stable biases and corresponding unstable biases between each stable bias;
a radiation detector configured to receive the radiation from the radiation source and to produce an electrical signal corresponding to the intensity of the received radiation;
data processing circuitry configured to acquire a first set of data from the electrical signal produced by the radiation detector when an activation signal is supplied and configured to acquire a second set of data from the electrical signal produced by the radiation detector when the activation signal is not supplied; and
a controller unit coupled to the radiation source and the data processing circuitry and configured to synchronize the application of the stable biases to the radiation source with the application of the activation signal to the data processing circuitry.

9. The system of claim 8, further comprising a grid configured to allow the radiation source to emit radiation only during the application of the stable biases.

10. The system of claim 8, further comprising a processor configured to process one or both of the first set of acquired data and the second set of acquired data to construct one or more computed tomography (CT) images.

11. The system of claim 10, wherein the processor uses the first set of acquired data, the second set of acquired data, or both, to construct one or more monochromatic CT images.

12. The system of claim 10, wherein the one or more CT images comprises at least one multi-energy CT image that is constructed from the first set of acquired data and the second set of acquired data using a weighted calculation.

13. The system of claim 12, wherein the weighted calculation comprises a weighted least squares estimate, a weighted average, or a weighted subtraction calculation.

14. A method of energy separation in a multi-energy switching X-ray imaging system comprising:
monitoring a source bias of a switching X-ray source as it is switched between a low bias and a high bias and emits X-rays;
detecting the emitted X-rays using an X-ray detector that produces an electrical signal corresponding to the detected X-rays;
activating data processing circuitry to acquire a first set of data from the detector when the source bias is stable at the low bias or at the high bias; and
processing the first set of acquired data with a processor to construct one or more multi-energy computer tomography (CT) images.

15. The method of claim 14, wherein the source bias is stable when the source bias is within 10% of a mean low bias or a mean high bias.

16. The method of claim 14, wherein the source bias is stable when the source bias changes by 10% or less within a length of time.

17. The method of claim 16, wherein the length of time is a multiple of an acquisition time interval of the detector or a fraction of a period of a function representing the source bias over time.

18. The method of claim 14, further comprising:
activating the data processing circuitry to acquire a second set of data from the detector when the source bias is unstable; and
processing the first set of acquired data with the processor, the second set of acquired data, or both, to construct one or more non-multi-energy CT images.

19. The method of claim 18, wherein the source bias is unstable when the bias is not within 10% of a mean low bias or a mean high bias.

20. The method of claim 18, wherein the source bias is unstable when the bias changes by more than 10% within a period of time and the period of time is a multiple of an acquisition time interval of the detector or a fraction of a period of a function representing the source bias over time.
